(19) United States
(12) Patent Application Publication
(10) Pub. No.: US 2025/0369019 A1
(43) Pub. Date: Dec. 4, 2025

(54) METHOD OF GENETICALLY RECOMBINING LACTIC ACID BACTERIA AND GRAM-POSITIVE BACTERIA USING CRISPR/CAS SYSTEM

(71) Applicant: Chungbuk National University Industry-Academic Cooperation Foundation, Cheongju-si (KR)

(72) Inventors: Nam Soo Han, Sejong Special Self-Governing City (KR); Yong Su Jin, Champaign, IL (US); Seul Ah KIM, Cheongju-si (KR); Mihn Tri Nguyen, Da Lat City (VN); Sung Hoon Hong, Seoul (KR)

(73) Assignee: Chungbuk National University Industry-Academic Cooperation Foundation, Cheongju-si (KR)

(21) Appl. No.: 19/220,652

(22) Filed: May 28, 2025

(30) Foreign Application Priority Data
May 29, 2024 (KR) 10-2024-0070285

(51) Int. Cl. C12N 15/90; C12N 1/20; C12N 9/22; C12N 13/00; C12N 15/113

(52) U.S. Cl. CPC C12N 15/902; C12N 1/20; C12N 9/226; C12N 13/00; C12N 15/113; C12N 2310/20; C12N 2310/315

(57) ABSTRACT

Disclosed is a method of genetically recombining lactic acid bacteria and gram-positive bacteria using the CRISPR/Cas system, more specifically, to a method of genetically recombining lactic acid bacteria and gram-positive bacteria that is capable of recombining efficiently lactic acid bacteria and gram-positive bacteria that are difficult to recombine by increasing the efficiency of the RNP recombination system using Cas proteins. However, there is a problem in which it is difficult to recombine genes of Gram-positive bacteria and lactic acid bacteria even using the CRISPR/Cas system due to the cell wall structure thereof. On the other hand, it was found that genes of lactic acid bacteria and gram-positive bacteria that are difficult to recombine can be recombined with high efficiency by using recombinases in combination with a phosphorothioated donor DNA in an RNP recombination system using Cas protein.

### CROSS-REFERENCE TO RELATED APPLICATIONS

This application claims the benefit of Korean Patent Application No. 10-2024-0070285, filed on May 29, 2024, the entire disclosure of which is incorporated herein by reference.

### INCORPORATION BY REFERENCE OF SEQUENCE LISTING

The content of the electronically submitted sequence listing, file name: Q309293_sequence listing as filed; size: 33,326 bytes; and date of creation: May 28, 2025, filed herewith, is incorporated herein by reference in its entirety.

### BACKGROUND OF THE INVENTION

### Field of the Invention

The present invention relates to a method of genetically recombining lactic acid bacteria and gram-positive bacteria using a CRISPR/Cas system, more specifically, to a method of genetically recombining lactic acid bacteria and gram-positive bacteria that is capable of efficiently recombining lactic acid bacteria and gram-positive bacteria that are difficult to recombine by increasing the efficiency of a RNP recombination system using Cas proteins.

### Description of the Related Art

Gram-positive bacteria refer to microorganisms whose cell walls are surrounded by peptidoglycan and play an important role in various industrial fields such as medicine, food, and the environment, depending on the characteristics thereof.

Lactic acid bacteria are Gram-positive bacteria that mainly produce lactic acid as a result of fermentation and are found in large quantities in fermented foods such as yogurt and kimchi. Lactic acid bacteria are generally known to be safe, and have been consumed in various forms of fermented foods for a long time, and contribute not only to the taste of fermented foods but also to health promotion. Lactic acid bacteria not only produce lactic acid during the fermentation process to provide acidity, but also produce aromatic components such as acetoin and diacetyl, and produce polysaccharides, contributing to physical properties. Lactic acid bacteria can also suppress harmful bacteria or pathogenic microorganisms that may cause diseases, and help maintain the balance of intestinal microorganisms.

Gene editing technology enables lactic acid bacteria to function more effectively in the human body and minimizes side effects or the risk of infection. In addition, the metabolic pathway can be changed by editing the genome of lactic acid bacteria to produce useful substances or to improve functions related to strengthening the immune system. In other words, the applicability of gene editing of lactic acid bacteria to the food and bio industries such as production of useful metabolites can be increased.

Meanwhile, CRISPR gene scissors (CRISPR/Cas9) have rapidly developed since they were first developed in 2013 and are widely used in various species such as human cells, animal cells, plant cells, yeast, and fungi. This method uses the CRISPR immune system formed by bacteria to prevent the invasion of external DNA as gene scissors. In other words, when a fragment of the base sequence (CRISPR part) that searches for the base sequence of a specific gene is produced and paired with the cleaving enzyme Cas9, the paired CRISPR system attaches to the target DNA base sequence and cleaves the same. CRISPR gene scissors have been confirmed to be the easiest to produce, among the scissors developed to date, the cheapest and to have high accuracy and efficiency.

Gene editing using CRISPR gene scissors is mainly performed by introducing CRISPR/Cas9 expression genes using a plasmid expression system. However, when the plasmid expression system is used, the Cas9 protein is constantly expressed in large quantities and thus is the probability of cutting unintended base sequence positions (off-target effect) is high. In addition, there is an inconvenience in which the system should be constructed again with a plasmid that is suitable for each type of host. In addition, there is a problem in which plasmid-based genome editing systems leave behind antibiotic marker DNA.

Therefore, a method of introducing CRISPR gene scissors into cells using sgRNA (guide RNA) and protein complexes (ribonucleoproteins, RNPs) without inserting external DNA (DNA-free) has been developed. Cas9 delivery using ribonucleoprotein complexes (RNPs) is a desirable method because it is easy to produce, has low off-target effects, and does not require a vector system. Because of these advantages, this RNP recombination system is being used for editing various eukaryotic cells such as animals, plants, fungi, yeast, and microalgae.

However, despite the advantages, the CRISPR gene scissors editing technology using the RNP recombination system has not yet been used in lactic acid bacteria and Gram-positive bacteria.

This is due to the following problems.

Lactic acid bacteria and Gram-positive bacteria have a complicated cell wall structure, making it difficult to introduce high concentrations of RNP into the cells. Electroporation is generally used as a method of delivering RNP into the cells. Since lactic acid bacteria and Gram-positive bacteria have a complicated cell wall structure, they are introduced by applying a high voltage of over 10,000 V/cm although competent cells are used. However, the results of research of the present inventors showed that the RNP-type Cas9 protein loses activity under such high voltage and the success rate of RNP transduction decreases under low voltage conditions.

In addition, in order to introduce genetic traits into the target DNA, the host must have a recombinase enzyme gene that acts to repair DNA cut by Cas9. While various eukaryotic cells such as animals, plants, fungi, yeast, and microalgae have the corresponding recombinase enzyme, lactic acid bacteria and gram-positive bacteria do not have or lack the recombinase gene, thus having a problem that homologous recombination occurs at an extremely low rate.

In addition, donor DNA (a DNA fragment to be introduced into the host chromosome) introduced along with RNP for homologous recombination is hydrolyzed by nuclease present in the cytoplasm of lactic acid bacteria and gram-positive bacteria and thus the possibility of the desired transduction is extremely low. Therefore, although RNP cuts the exact target location of the lactic acid bacteria chromosome, there is a problem that the probability of donor DNA being introduced through recombination is extremely low.

### PRIOR ART LITERATURE

### Patent Literature

(Patent literature 1) Korean Patent Publication No. 10-2021-0123237 (Oct. 13, 2021) discloses a gene editing method based on the CRISPR/Cas9 system.

(Patent literature 2) Korean Patent Publication No. 10-2022-0124652 (Sep. 14, 2022) discloses a gene editing method based on the CRISPR/Cas9 system.

### SUMMARY OF THE INVENTION

Therefore, the present invention has been made in view of the above problems, and it is an object of the present invention to provide a method for highly efficiently recombining genes of lactic acid bacteria and gram-positive bacteria, which are difficult to recombine, by increasing the efficiency of an RNP recombination system using a Cas protein.

In accordance with an aspect of the present invention, the above and other objects can be accomplished by the provision of a method of recombining genes in Gram-positive bacteria cells by introducing a ribonucleoprotein (RNP) complex in which a site-specific endonuclease and a guide RNA (gRNA) that specifically binds to a target DNA and guides a cleavage site of the site-specific endonuclease are linked, and a donor DNA into the Gram-positive bacteria cells, wherein recombinases are introduced into the cells along with the ribonucleoprotein and the DNA donor, and the DNA donor has a phosphorothioate structure in which at least one oxygen of the phosphate backbone structure is substituted with sulfur.

The site-specific endonuclease may include one selected from Cas3, Cas9, Cas12, Cas12a (Cpf1), Cas13, Cas14, and nickase variants thereof.

The recombinant enzyme may include one or more selected from RecT, RecE, RedE, RedT, RamdaE, and RamdaT.

The ribonucleoprotein may include the site-specific endonuclease and the guide RNA (gRNA) in a molar ratio of 1:0.8 to 1:1.2.

The cell may be a competent cell that has a weakened cell wall due to being cultured along with at least one selected from penicillin, ethanol, glycine, and sodium chloride (NaCl).

The cell may be a lactic acid bacterium.

The DNA donor may have a phosphorothioate structure in which oxygen of the phosphate backbone structure at both ends is substituted with sulfur.

The introduction may be performed by electroporation. The electroporation may be performed at a voltage of 8 to 12 kV/cm.

In accordance with another aspect of the present invention, there is provided a cell genetically recombined by the method.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows the results of SDS-PAGE analysis to determine whether or not a cas9 protein was produced intact, after the cas9 protein (159 kDa) was produced and purified and;

FIG. 2 shows the results of size analysis to determine whether or not four synthesized sgRNAs (DS52, DS182, DS365, DS433) with a size of 123 bp targeting the dsr (dextransucrase) gene of Leuconostoc citreum were produced intact;

FIG. 3 shows the results of determination as to whether or not cleavage occurred after treating the dextransucrase (dsr) gene amplicon of L. citreum with Cas9/sgRNA RNP and in order to determine whether or not the produced sgRNAs (DS52, DS182, DS365, DS433) are fully active. The results showed that, among the four sgRNAs, DS365 and DS433 successfully cleaved the target dsr gene fragment and in subsequent experiments, DS365 and DS433 were used for RNP production;

FIG. 4 shows the results obtained by treating Cas9/sgRNA RNP with the dsr gene amplicon of L. citreum (A) and quantifying the cleavage efficiency using the ImageJ program (B) in order to evaluate the RNP cleavage efficiency depending on the sgRNA concentration of Cas9/sgRNA RNP. The results showed that the highest DNA cleavage efficiency was obtained when Cas9 and sgRNA were mixed at approximately the same molar ratio (1:1) during the RNP preparation process;

FIG. 5 shows the results of evaluation of the cleavage activity of RNP after applying an electric field of 8 to 12 kV/cm to Cas9/sgRNA RNP in order to determine the effect of the electric field on the activity of Cas9/sgRNA RNP. The results showed that the relative efficiency of Cas9/sgRNA RNP rapidly decreases when a voltage of 10 kV/cm or higher is applied;

FIG. 6 shows the results of SDS-PAGE analysis to determine whether or not the RecT protein was produced intact in a soluble form after cloning the RecT protein (34 kDa) from Lactobacillus plantarum WCFS1 and expressing the genes in large quantities in E. coli BL21 harboring the pET21a-RecT-His vector, followed by purification;

FIG. 7 shows the results of SDS-PAGE analysis to determine whether or not the RecE protein was produced intact in a soluble form after cloning the RecE protein (36 kDa) from E. coli and expressing the genes in large quantities in E. coli BL21 harboring the pET28b-RecE-His vector, followed by purification;

FIG. 8 is a schematic diagram illustrating a process of constructing phosphothioated donor DNA through overlap extension PCR (A), and the result of determination as to whether or not the phosphothioated donor DNA was completely synthesized (B);

FIG. 9 shows the result of determination as to whether or not the phosphorothioated donor DNA or general donor DNA was degraded by nucleases inside the cytoplasm after the lactic acid bacteria to which phosphorothioated donor DNA or general donor DNA to be introduced was mixed with the cytoplasmic fragment, in order to determine the characteristics of the phosphorothioated donor DNA. The result shows that the phosphorothioated donor DNA was not degraded by the introduced cytoplasmic nuclease (A), whereas general donor DNA was mostly degraded within 30 minutes (B);

FIG. 10 shows the result of determination as to whether or not circular plasmid or linear DNA was degraded after mixed with RecE protein;

FIG. 11 shows the results observed after transformation using cells at various concentrations ((A) 10⁵⁻⁶/mL, (B) 10^{2˜3}/mL, (C) 10⁴⁻⁵/mL) in order to determine the appropriate cell concentration used to transform the strain;

FIG. 12 shows the results of the viable cell concentration obtained by introducing cas9, sgRNA, and RNP into cells or L. citreum competent cells treated with penicillin, and then culturing the same in order to determine the improved introduction efficiency of competent cells whose cell wall synthesis is inhibited by treatment with penicillin, and the results of evaluation of the DNA double-strand cleavage efficiency through cell death;

FIGS. 13 and 14 show the results obtained by transformation of L. citreum competent cells, whose cell wall synthesis was inhibited by treatment with penicillin, using Cas9/sgRNA RNP, phosphorothioated donor DNA or donor DNA, and RecT, followed by separation;

FIG. 15 shows the results of comparison of the viable cell concentration from the results of FIGS. 13 and 14, respectively, which indicates that RecT acts as a protector during electric shock, thus reducing the number of dead cells;

FIG. 16 is an SEM image obtained after culturing in a sucrose-containing medium to distinguish between L. citreum EFEL2703 (mutant strain) and L. citreum EFEL2700 (wild-type strain). The wild-type strain produces dextran polysaccharide in sucrose medium (B), whereas the knockout mutant strain with the dextran sucrase gene does not produce dextran polysaccharide in sucrose medium (D). These results indicate that the wild-type strain and the mutant strain can be distinguished by polysaccharide formation;

FIG. 17 shows the results of determination of the effect of increasing transformation efficiency depending on the use of phosphorothioated donor DNA and recombinant enzyme of L. citreum competent cell;

FIG. 18 shows the results of determination of the size of the dsr (dextransucrase) gene amplification product of L. citreum EFEL2703 (mutant strain) and L. citreum EFEL2700 (wild type strain) in order to determine whether or not transformation occurred completely;

FIG. 19 shows the results of determination of the expression of the 225 kDa dsr (dextransucrase) gene of L. citreum EFEL2703 (mutant strain) and L. citreum EFEL2700 (wild type strain) to determine whether or not transformation occurred completely;

FIG. 20 shows the results of determination as to whether or not dextran polysaccharide was formed after culturing L. citreum EFEL2703 (mutant strain) and L. citreum EFEL2700 (wild type strain) in MRS medium containing sucrose, to determine whether or not transformation was complete;

FIG. 21 shows the results of measurement of the size to determine whether or not synthesized sgRNA (upp182, upp212) targeting the upp (uracil phosphoribosyltransferase) gene of Bifidobacterium bifidum BGN4 was completely produced;

FIG. 22 shows the results of determination as to whether or not cleavage occurred after the upp gene amplicon of Bifidobacterium bifidum BGN4 was treated with Cas9/sgRNA RNP to determine whether or not the produced sgRNA (upp182, upp212) was completely active;

FIG. 23 shows the results of determination as to whether or not phosphorothioated donor DNA was completely synthesized by overlap extension PCR;

FIG. 24 shows the results obtained by plating Bifidobacterium bifidum BGN4 cells on a general MRS medium, followed by culturing;

FIG. 25 shows the results obtained by plating Bifidobacterium bifidum BGN4 cells on a general MRS medium containing 5-FU (5-fluorouracil), followed by culturing, in order to screen transformed strains with a deletion of the UPP (uracil phosphoribosyltransferase) gene;

FIG. 26 shows the results of determination of the DNA cleavage efficiency by RNP using the viable cell concentration obtained using FIG. 24;

FIG. 27 shows the transformation efficiency depending on the introduction of phosphorothioated DNA and RecE/T recombinant enzyme using the viable cell concentration obtained in FIG. 25;

FIG. 28 shows the results of determination of the size after amplifying the upp gene of the Bifidobacterium bifidum BGN4 wild-type strain and mutant strain to determine whether or not the transformation occurred completely; and

FIG. 29 shows the results of comparing the upp gene sequences of the Bifidobacterium bifidum BGN4 wild-type strain and mutant strain to determine whether or not the transformation occurred completely.

### DETAILED DESCRIPTION OF THE INVENTION

The present invention provides a method of recombining genes in Gram-positive bacteria cells by introducing a ribonucleoprotein (RNP) complex in which a site-specific endonuclease and a guide RNA (gRNA) that specifically binds to a target DNA and guides a cleavage site of the site-specific endonuclease are linked, and a donor DNA into the Gram-positive bacteria cells, wherein recombinases are introduced into the cells along with the ribonucleoprotein and the DNA donor, and the DNA donor has a phosphorothioate structure in which at least one oxygen of the phosphate backbone structure is substituted with sulfur.

Gene editing using CRISPR gene scissors has been used for editing various eukaryotic cells such as animals, plants, fungi, yeast, and microalgae, but CRISPR gene scissors editing technology using the RNP recombination system has not been used in lactic acid bacteria and Gram-positive bacteria for the following reasons.

Lactic acid bacteria and Gram-positive bacteria have a complicated cell wall structure, making it difficult to introduce high concentrations of RNP into the cells. When lactic acid bacteria and Gram-positive bacteria are introduced by electroporation, a high voltage of over 10,000 V/cm should be applied due to the complicated cell wall structure thereof, although competent cells are produced. However, in this case, the RNP may lose activity due to such high voltage.

In addition, donor DNA (a DNA fragment to be introduced into the host chromosome) introduced along with RNP for homologous recombination is hydrolyzed by nuclease present in the cytoplasm of lactic acid bacteria and gram-positive bacteria and thus the possibility of the desired transduction is extremely low.

However, in the present invention, when genes are edited based on the CRISPR gene scissors editing technology using the RNP recombination system, the efficiency of editing genes can be maximized by further introducing recombinases into cells and using a phosphorothioated donor DNA including a phosphorothioate structure. The result showed that a desired gene sequence can be inserted by recombining genes of Gram-positive bacteria and lactic acid bacteria, which are difficult to edit.

Meanwhile, in the present invention, the site-specific endonuclease refers to a Cas protein in the gene editing technology using CRISPR gene scissors. The Cas protein acts along with guide RNA (gRNA) and exhibits cleavage activity at a specific gene site. Examples of Cas proteins include Cas3, Cas9, Cas12, Cas12a (Cpf1), Cas13, Cas14, and nickase variants thereof. Among them, Cas9 and Cpf1, which have simple structures and systems, are most commonly used for gene editing.

In the present invention, guide RNA (gRNA) functions to guide Cas protein to target DNA by binding to site-specific endonuclease. Guide RNA may vary depending on the type of site-specific endonuclease, but may be composed of, for example, crRNA (CRISPR RNA) that recognizes and binds to a specific DNA sequence and tracrRNA (trans-activating crRNA) that links the crRNA to Cas protein.

Here, the recombinase refers to a general term for proteins that cut, release, and combine DNA to increase repair efficiency, and preferably refers to a protein involved in recombination. The recombinase may include, for example, at least one selected from RecT, RecE, RedE, RedT, RamdaE, and RamdaT. In the following examples, RecT and RecE were used as examples of recombinant enzymes and it was confirmed that recombination efficiency was further increased using the recombinant enzyme. RecT acts as a single strand annealing protein and promotes the combination of complementary DNA strands, and RecE is an exonuclease that acts on DNA double strands and is an enzyme that creates a DNA overhang in which the 3′-terminus is a single strand. Lactic acid bacteria have a problem in that homologous recombination is not performed completely. However, it was found that, when a recombinant enzyme is further introduced according to the present invention, homologous recombination can be performed completely.

In the present invention, ribonucleoprotein (RNP) means an RNA-protein complex in which RNA and protein are linked, and in the present invention, ribonucleoprotein (RNP) means an RNA-protein complex produced by linking a site-specific endonuclease to guide RNA (gRNA). Meanwhile, the following example showed that it is preferable to use site-specific endonuclease and guide RNA (gRNA) in a molar ratio of 1:0.8 to 1:1.2 in the genetic recombination of gram-positive bacteria or lactic acid bacteria.

In the present invention, the DNA donor (donor DNA) refers to a sequence inserted into a target gene, and examples thereof may include a polynucleotide, a gene sequence, a translation control sequence, a signal sequence, a promoter, a terminator sequence, an mRNA sequence, and the like.

Meanwhile, the present invention is characterized by using a phosphorothioated donor DNA including a phosphorothioate structure in which the oxygen of the phosphate backbone structure is replaced with sulfur. The following examples showed, when a phosphorothioated donor DNA is used, the resistance to hydrolysis, that is, the “stability of the donor DNA”, is increased and the recombination efficiency is further increased. Meanwhile, the phosphorothioated donor DNA preferably includes a phosphorothioate structure in the sequence of 1 to 5 bp from both ends.

In the present invention, the cell refers to a cell that is the target of genetic recombination. Meanwhile, it is known that it is difficult to recombine genes using the cas protein system in prokaryotic cells having thick cell walls, such as gram-positive bacteria and lactic acid bacteria. However, it has been found that, when the method of the present invention is applied, genetic recombination can be performed with high efficiency even in cells with thick cell walls.

Meanwhile, the cell used in the cell genetic recombination method of the present invention is preferably a competent cell with a weakened cell wall which is cultured with at least one substance selected from penicillin, ethanol, glycine, and sodium chloride (NaCl). According to the following examples, when recombination is performed, the amount of ribonucleoprotein (RNP), donor DNA, and recombinases introduced into the cell wall increases, enabling transformation with higher efficiency.

Meanwhile, in the present invention, the introducing ribonucleic acid proteins (RNPs), donor DNA, and recombinases into cells may be, for example, performed using an electroporation method. Meanwhile, when genetic recombination is performed in cells with thick cell walls such as Gram-positive bacteria or lactic acid bacteria, it is preferable to introduce ribonucleic acid proteins (RNPs) using electroporation by applying a high-power electric field such as 8 to 12 kV/cm.

Meanwhile, the cell genetic recombination method of the present invention is preferably performed by mixing 10³⁻⁶/mL of cells, 100 to 160 μg of RNP (molar ratio of Cas9:sgRNA=1:0.8 to 1:1.2), 10 to 20 μg of RecT, 10 to 20 μg of RecE, and 10 to 20 μg of phosphorothioated donor DNA, and then introducing the same into cells using electroporation to perform transformation.

Hereinafter, the present invention will be described in more detail with reference to the following examples, but the scope of the present invention is not limited to the examples and includes variations and technical concepts equivalent thereto.

### EXAMPLE 1: PRODUCTION OF Cas9/sgRNA RNP

### 1-1. Production of Cas9 Protein

Cas9 protein was produced using E. coli BL21 (DE3) transformed with the pET-Cas9-NLS-6His plasmid commercially available from Addgene (USA).

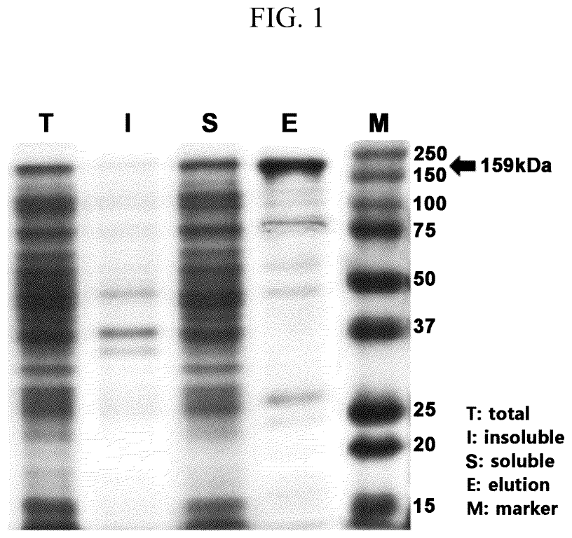

E. coli was inoculated into 200 mL of LB medium containing 50 μg/mL kanamycin and cultured at 37° C. and 200 rpm. When the optical density (600 nm) reached 0.4 to 0.5, and 1 M IPTG was added at a concentration of 0.8 mM, followed by further culturing at 18° C. and 200 rpm overnight to induce expression. The cells were harvested by centrifugation at 6,000×g for 30 min at 4° C. and resuspended in 10 mL of lysis buffer (pH 7.4, 50 mM NaH₂PO₄, 300 mM NaCl, 10 mM imidazole) supplemented with 1 mM PMSF. The cells were disrupted on ice using an ultrasonicator at 33% amplitude for 10 min under conditions of ON for 10 sec/OFF for 10 sec. The disrupted cells were centrifuged at 12,000×g and at 4° C. for 30 min, and the supernatant was allowed to pass through a 0.45 μm syringe filter. Then, the Cas9 protein (159 kDa) was purified using a Ni-NTA agarose column and concentrated using an Amicon Ultra centrifugal filter (30K MWCO). The purified Cas9 protein was stored in storage buffer (150 mM NaCl, 20 mM HEPES, 0.1 mM EDTA, 1 mM DTT, 2% sucrose, 20% glycerol) and used in the following examples (FIG. 1).

### 1-2. Production of sgRNA

sgRNA was produced using an in vitro synthesis method to insert donor DNA into the DSR gene sequence. sgRNA candidates (DS52, DS182, DS365, DS433) were selected using the dextransucrase (DSR) gene sequence (SEQ ID NO: 1) of L. citreum EFEL2700 (KACC 91348P). Then, sgRNA was produced using oligomer F (DS52, DS182, DS365, DS433) and oligomer R bound with 23 bases added according to Table 1 below.

Specifically, two types of oligomers were extended for 30 cycles using GeneAmp PCR system 2400 (Applied Biosystems) with Pfu polymerase (Thermo Fisher). After extension, sgRNA DNA templates were purified using AccuPrep PCR/Gel purification kit (Bioneer, Daejeon, Korea).

TABLE 1

| | | SEQ ID |
|---|---|---|
| Primer | Sequence | NO. |
| Oligo | GAAATTAATACGACTCACTAT | 2 |
| F(DS52) | AGTCTACAAGTCTGGTAAGAG | |
| | TGTTTTAGAGCTAGAAATAGC | |
| | AAG | |
| Oligo | GAAATTAATACGACTCACTAT | 3 |
| F(DS182) | AGAACGACAGTATCGTGTTGA | |
| | TGTTTTAGAGCTAGAAATAGC | |
| | AAG | |
| Oligo | GAAATTAATACGACTCACTAT | 4 |
| F(DS365) | AGCGTAACAGTCGGCAACTGC | |
| | TGTTTTAGAGCTAGAAATAGC | |
| | AAG | |
| Oligo | GAAATTAATACGACTCACTAT | 5 |
| F(DS433) | AGATCTGGAAAAAGATGGTAA | |
| | AGTTTTAGAGCTAGAAATAGC | |
| | AAG | |
| Oligo | AAAAAAGCACCGACTCGGTGC | 6 |
| R(common) | CACTTTTTCAAGTTGATAACG | |
| | GACTAGCCTTATTTTAACTTG | |
| | CTATTTCTAGCTCTAAAAC | |

RNA transcription was performed on the purified sgRNA DNA template using a T7 RNA polymerase under the conditions in Table 2.

TABLE 2

| Component | Volume (μL) |
|---|---|
| Template DNA | 3~5 (1,560 ng) |
| 50 mM MgCl₂ | 28 |
| dNTP mixture (100 mM stock) | 16 |
| 10x T7 RNA buffer | 10 |
| T7 RNA polymerase (50 U/μL) | 5 |
| RNase inhibitor (40 U/μL) | 2.5 |
| DEPC-treated water | up to 100 |

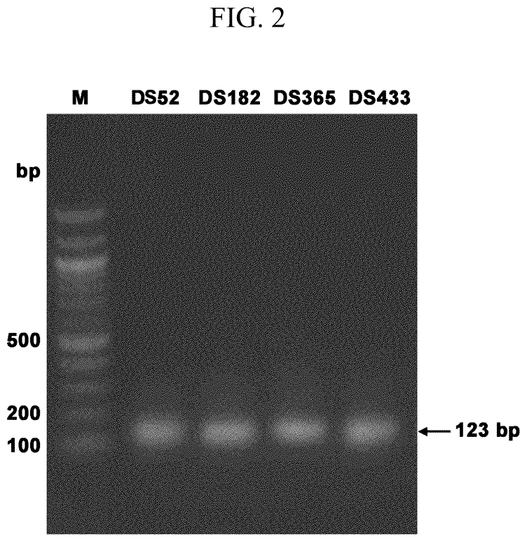

Then, the sgRNA DNA template was removed by treatment with 1 μL of DNase I (2 U/μL) at 37° C. for 1 hour, and sgRNA was purified using an RNA purification kit (NEB, MA, USA). Then, sgRNA was identified by agarose electrophoresis. The result showed that the expected 123 bp band was successfully observed in all sgRNA candidates after transcription. Then, the purified RNA was stored at −80° C. and used in the following examples (FIG. 2).

### 1-3. Production of Ribonucleoprotein (RNP)

500 ng of Cas9 protein and 250 ng of sgRNA were mixed and reacted at room temperature for 15 minutes to assemble Cas9/sgRNA RNP.

To determine whether or not the sgRNA of RNP can completely cleave the gene of L. citreum, the PCR amplicon was prepared by amplifying the gDNA of L. citreum EFEL2700 as a template through PCR using the DSR-U-F primer (5′-CAGCTAAACTCACTTTAACTATTGCT-3′, SEQ ID NO: 9) and the DSR-D-R primer (5′-CTAGGCATGTTGTATTGTGTATATT-3′, SEQ ID NO: 12).

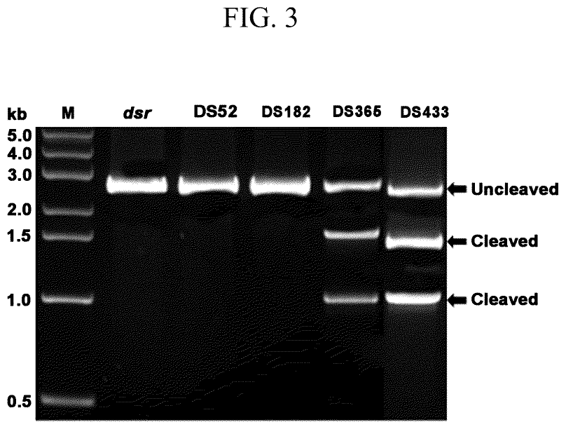

Then, the RNP was reacted with 100 to 150 ng of the PCR amplicon at 37° C. for 1 hour to test whether or not the RNP could fully exhibit activity. After the reaction was completed, the reaction was stopped by adding a stop solution (30% glycerol, 1.2% SDS, 250 mM EDTA (pH 8.0)), and then analyzed on a 2% agarose gel (FIG. 3).

As can be seen from FIG. 3, the DNA of the PCR amplicon was cleaved in the group using the gDS365 and gDS433 sgRNA candidates. The result showed that gDS365 and gDS433 sgRNAs were sgRNAs suitable for transformation. Therefore, gDS365 and gDS433 sgRNAs were used in the following examples.

### 1-4. Optimization of Concentration of Produced Ribonucleoprotein (RNP)

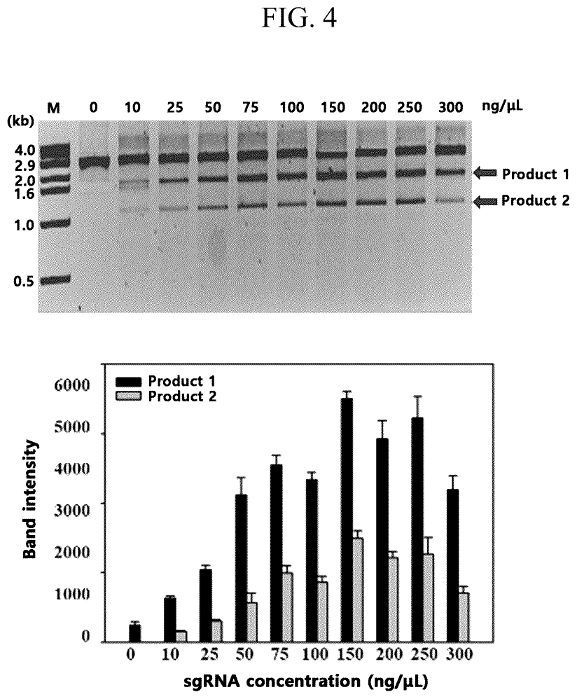

To determine the optimal concentration conditions for RNP production, treatment with sgRNA gDS365 and gDS433, whose cleavage efficacy had been found to be good, was performed at concentrations ranging from 0 to 300 ng/μL, while the amount of Cas9 protein was fixed, to assemble Cas9/sgRNA RNP, and the activity thereof was determined by treating the PCR amplicon with Cas9/sgRNA RNP (FIG. 4).

As can be seen from FIG. 4, the thickest band appeared in the group treated with 150 ng/μL of sgRNA. The result showed that the RNP could exhibit the highest activity when Cas9 and sgRNA was mixed at a molar ratio of 1:1.

### 1-5. Confirmation of the Effect of Electric Field on RNP

Electroporation is an efficient technique for delivering a genetic material to lactic acid bacteria, but the activity of Cas9 RNP may be affected by the electric field and thus the efficiency of genome editing may decrease. In this example, whether or not RNP affected by the electric field can function completely in cells was determined.

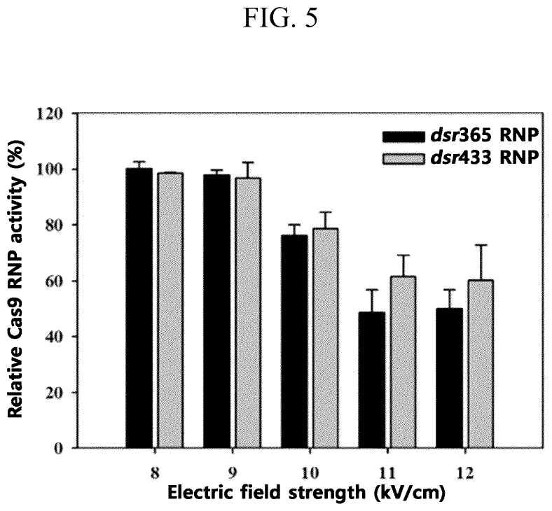

Cas9 protein and sgRNA were mixed in a molar ratio of 1:1 to form Cas9/sgRNA RNP, an electric field of 8 to 12 kV cm was applied to the complex, and the cleavage efficiency was determined (FIG. 5).

It is known that, when transformation is performed by introducing recombinases into cells using an electric field, the transformation efficiency increases as the voltage increases. However, it can be seen from FIG. 5 that the cleavage efficiency of RNP decreased as the voltage increased. This supports that application of an electric field of 10 kV/cm or more may affect the activity of RNP. However, since the transformation efficiency increases as the voltage increases, the voltage was set at 8 to 12 kV/cm in the following experiments.

### Example 2: Production of Recombinant Enzyme

In this example, RecT and RecE recombinant enzymes were produced to improve the efficiency of genetic recombination using the RNP recombination system.

### 2-1. Production of RecT Recombinant Enzyme

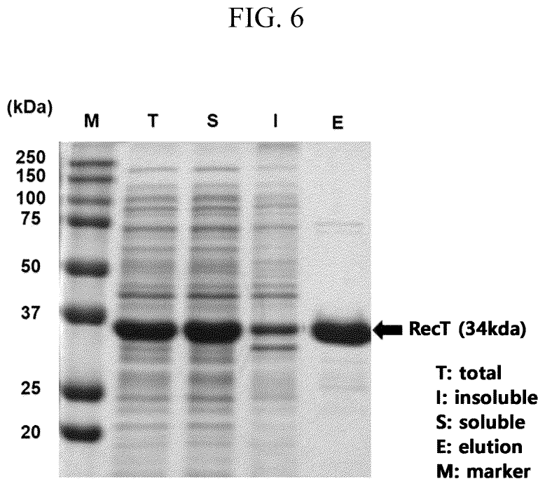

The RecT gene (lp_0641, SEQ ID No.: 7) derived from Lactobacillus plantarum WCFS1 was cloned into E. coli BL21 (DE3) using the pET-21a vector. Then, for gene expression, the E. coli BL21 variant was cultured in LB broth containing 0.8 mM IPTG and 100 μg/ml ampicillin at 37° C. for 15 hours. Then, the cells were disrupted with an ultrasonicator and the RecT recombinant enzyme (34 kDa) was purified through Ni-NTA affinity chromatography (FIG. 6).

### 2-2. RecE Recombinant Enzyme Production

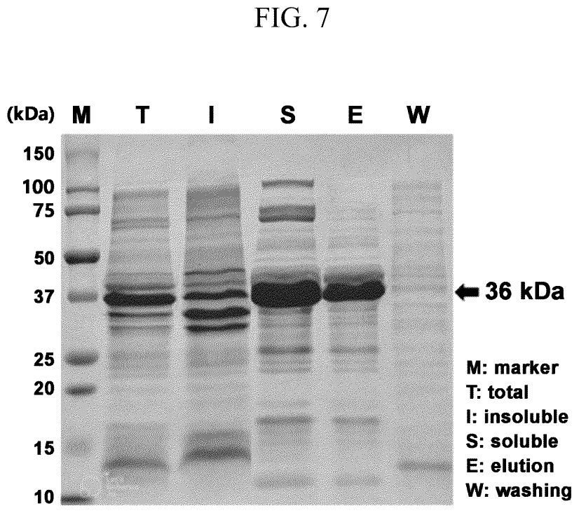

Plasmid pET28b-RecE-His expressing the RecE gene (SEQ ID NO: 8) derived from E. coli was cloned into E. coli BL21. Then, for gene expression, the E. coli BL21 variant was cultured in LB broth containing 0.8 mM IPTG and 100 μg/ml ampicillin at 37° C. for 15 hours. Then, the cells were disrupted by an ultrasonicator and RecE recombinant enzyme (36 kDa) was purified through Ni-NTA affinity chromatography (FIG. 7).

### Example 3: Production of Phosphorothioated Donor DNA

In this example, donor DNA and the phosphorothioated donor DNA, for use in gene recombination using an RNP recombination system, was produced. Meanwhile, donor DNA and phosphorothioated donor DNA were produced using conventional DSR gene sequences and were produced such that dextransucrase (DSR) would not be fully expressed even if a part of the DSR gene was inserted.

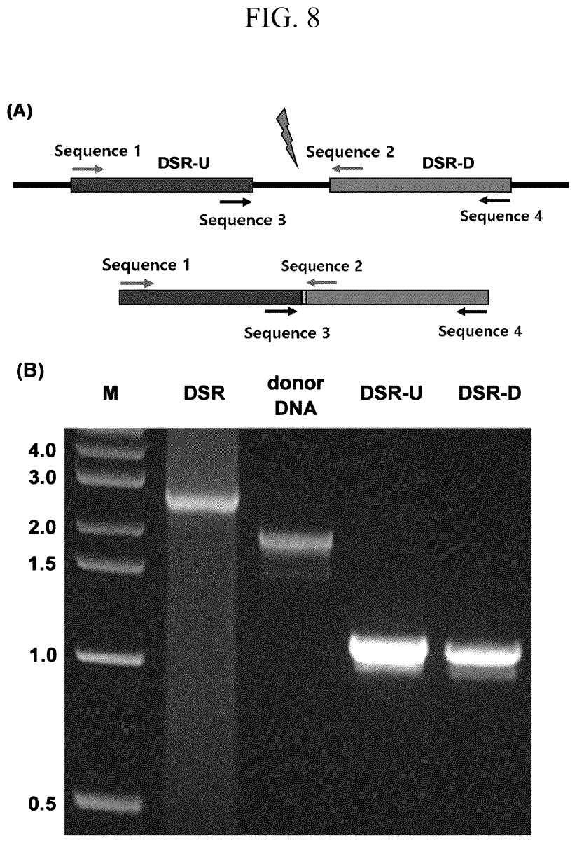

Specifically, upstream primers (DSR-U-F/DSR-U-R/phosphorothioated DSR-U-F) and downstream primers (DSR-D-F/DSR-D-R/phosphorothioated DSR-D-R) designed to delete a part of the DSR gene were produced (Table 3) and then DSR-U (1.0 kb) and DSR-D (1.1 kb) were amplified such that a 30 bp overlapping sequence was present at each end. Then, the amplified DSR-U and DSR-D primers were linked to each other by overlap extension PCR to produce donor DNA and phosphorothioated donor DNA ((B) of FIG. 8).

As can be seen from (B) of FIG. 8, when the existing gene of DSR was amplified, a band appeared at 2.7 kb, but in donor DNA and phosphorothioated donor DNA, a band appeared at 2.1 kb, which indicates successful production.

TABLE 3

| | | SEQ |
|---|---|---|
| | | ID |
| Name | Sequence | NO. |
| DSR-U-F | CAGCTAAACTCACTTTAA | 9 |
| | CTATTGCT | |
| DSR-U-R | GTCCACCGACTGTTCCCA | 10 |
| | GACTGTAGCTCTGTCGTT | |
| | GTGTCTGATGCTTTTACT | |
| DSR-D-F | AGAGCTACAGTCTGGGAA | 11 |
| | CAGTCGGTGGACGAAGCA | |
| | ACAACAGCTAATGACTTC | |
| DSR-D-R | CTAGGCATGTTGTATTGT | 12 |
| | GTATATT | |
| Phosphorothioated | *C*A*GCTAAACTCACT | 13 |
| DSR-U-F | TTAACTATTGCT | |
| Phosphorothioated | *C*T*AGGCATGTTGTA | 14 |
| DSR-D-R | TTGTGTATATT | |

*means phosphorothioated DNA between nucleotides

### Example 4: Characterization of RecE Protein

The recombination method of the present invention uses an additional RecE recombinant enzyme to further increase the recombination efficiency. At this time, whether or not the additionally used RecE could affect the donor DNA and rather decrease the recombination efficiency was determined.

### 4-1. Treatment of Phosphorothioated Donor DNA With RecE Protein

In order to determine the effect of increasing the recombination efficiency of phosphorothioated donor DNA, linear phosphorothioated DNA and linear general DNA were reacted with RecE protein and the degradation rates were compared.

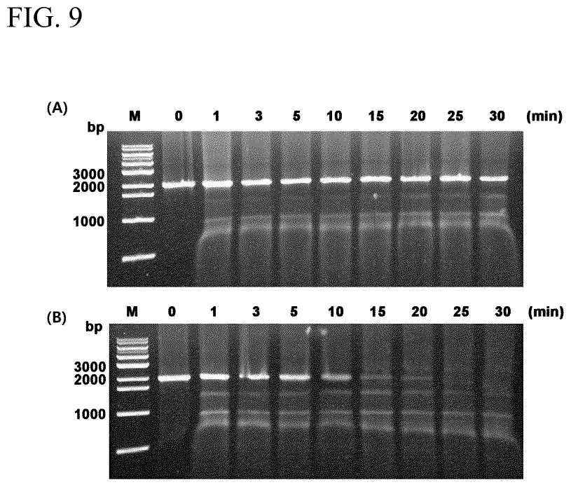

Specifically, linear phosphorothioated DNA and linear general DNA were produced by amplifying 2.7 kb of the dextransucrase (DSR) gene of L. citreum EFEL2700 strain, respectively. Then, linear DNA was reacted with the phosphorothioated linear DNA in a buffer containing RecE at a concentration of 100 nM, respectively, at 37° C. for 0, 10, and 30 minutes, and the reaction was stopped by adding a stop solution. Then, 1.5% agarose gel electrophoresis was performed for analysis (FIG. 9).

As can be seen from FIG. 9, the band shape of linear general DNA was broken and decomposed, whereas linear phosphorothioated DNA was not decomposed for up to 30 minutes. This indicates that, when general donor DNA is used, RecE recombinant enzyme may have an effect and thus lower recombination efficiency, whereas phosphorothioated donor DNA can be stably preserved in cells for a long time, exhibits a synergistic effect and thus increase recombination efficiency.

### 4-2. Treatment of Plasmid With RecE Protein

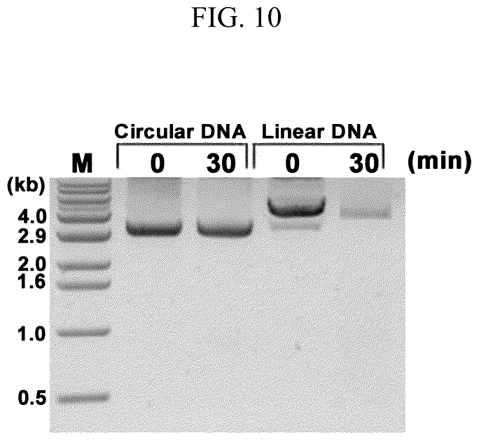

Whether or not RecE enzyme also affects plasmid was determined. For this purpose, the circular pMD20 plasmid and the linear pMD20 plasmid prepared by treating with PstI restriction enzyme were treated with RecE enzyme, the reaction was induced for 0 and 30 minutes at 37° C. in a buffer containing 100 nM RecE, and the reaction was stopped by adding a stop solution thereto. Then, analysis was performed by 1.5% agarose gel electrophoresis (FIG. 10).

As can be seen from FIG. 10, the circular plasmid DNA was not affected by RecE, whereas the linear plasmid DNA was affected by RecE and the thickness of the band decreased.

### Example 5: Confirmation of Appropriate Cell Concentration for Transformation

In this example, the concentration of cells used in the transformation process was optimized. In addition, whether or not the transformation efficiency could be increased when the cell wall was weakened by treatment with penicillin was determined.

2% L. citreum was inoculated into 30 mL of MRS medium, and when the OD600 of the culture reached 0.2, penicillin G (0.8 μg/mL) was added thereto to weaken the cell wall, and culture was further performed until OD600 reached 0.5. Then, the cells were harvested and resuspended in a solution for electroporation (EPS; 0.5 mol L-1 sucrose, 1 mmol L-1 K₂HPO₄·KH₂PO₄, and 1 mmol L⁻¹ MgCl₂, pH 7.4) at concentrations of 10²⁻³/mL, 10³⁻⁴/mL, 10⁴⁻⁵/mL, and 10⁵⁻⁶/mL to produce competent cells. The competent cells were stored at −80° C. and used in the following experiments.

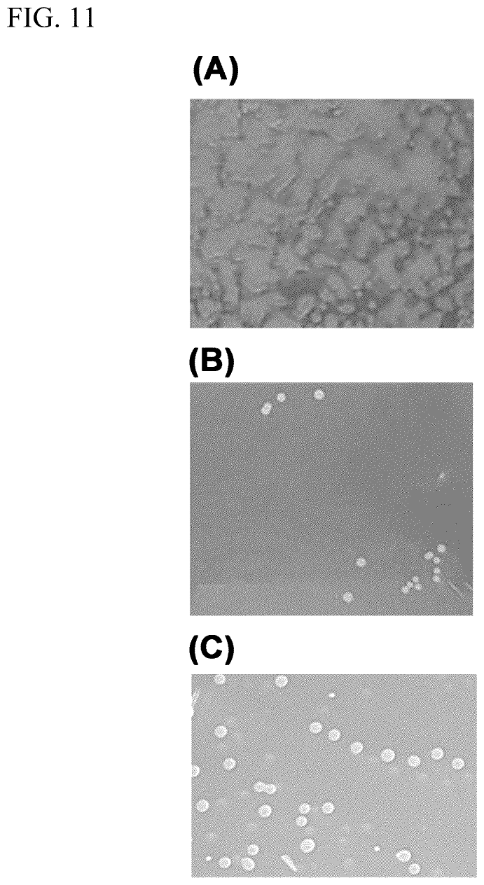

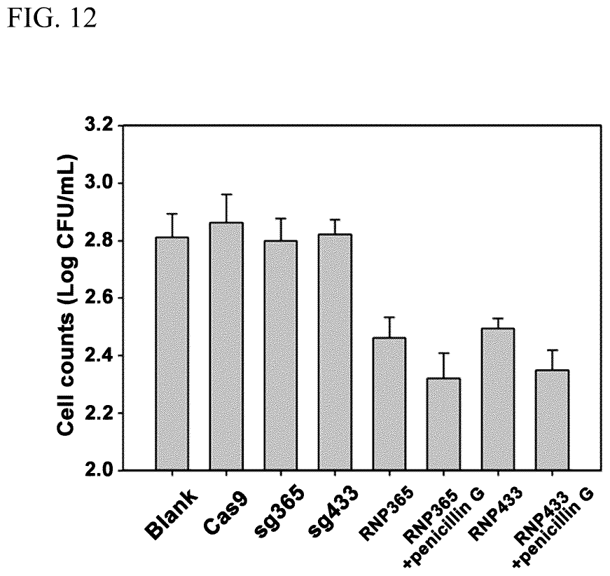

First, the effective concentration of transformed cells was determined. 20 μL of the competent cells at various concentrations (10^{2˜3}/mL, 10⁴⁻⁵/mL, 10⁵⁻⁶/mL) prepared above were mixed with RNP (Cas9:sgRNA=100 μg:30 μg), 24 μg of RecT, and 25 μg of phosphorothioated donor DNA, and then intracellular introduction was attempted using the electroporation (10 kV cm⁻¹, 25 μF, 400Ω) method. Then, the cells were plated on phenylethyl alcohol agarose plates (PES) containing 2% sucrose and cultured to measure the transformation efficiency (FIGS. 11 and 12).

FIG. 11 shows the results of observation of colonies formed in the medium, and the transformation efficiency was determined depending on whether or not polysaccharides were formed. In the experimental group (A) using competent cells at a concentration of 10⁵⁻⁶/mL, the relative RNP concentration was low, indicating that the transformation efficiency was not high. On the other hand, in the experimental group (B) using competent cells at a concentration of 10²⁻³/mL, the number of cells was small, indicating that the probability of obtaining mutant colonies was low. This shows that the cells used at a concentration of 10⁴⁻⁵/mL, like in the experimental group (C) using the competent cells at a concentration of 10⁴⁻⁵/mL, was the most suitable for the transformation process.

FIG. 12 shows the viable cell concentration obtained by introducing one or more selected from cas9, sgRNA, and RNP into cells at a concentration of 10⁴⁻⁵/mL or competent cells treated with penicillin, culturing the same on phenylethyl alcohol agarose plates, and counting the number of colonies formed in the medium.

When DNA is cleaved due to the endonuclease activity of RNP, the cell dies. As can be seen from FIG. 12, in the group using RNP, the viable cell concentration was reduced to 20-40% due to the cleavage of the DNA double strand by RNP. This shows that the DNA cleavage efficiency was 60-80%. In particular, the viable cell concentration was lower in the experimental group whose cell wall was weakened by treatment with penicillin. This shows that the efficiency of intracellular introduction using electroporation may be increased by weakening the cell wall.

### Example 6: Transformation for L. citreum Strain

### 6-1. Transformation

20 μL of the L. citreum EFEL2700 competent cell solution obtained at a concentration of 10⁴⁻⁵/mL by treating with penicillin obtained through Example 5 was electroporated (10 kVcm⁻¹, 25 μF, 400Ω) using 25 μg of RNP (Cas9:sgRNA=100 μg:30 μg=1:1 molar ratio) and phosphorothioated donor DNA or donor DNA, and 24 μg of RecT to perform transformation. Meanwhile, the resulting dsr inactive mutant was called “L. citreum EFEL2703”.

### 6-2. Screening of Mutant Strains

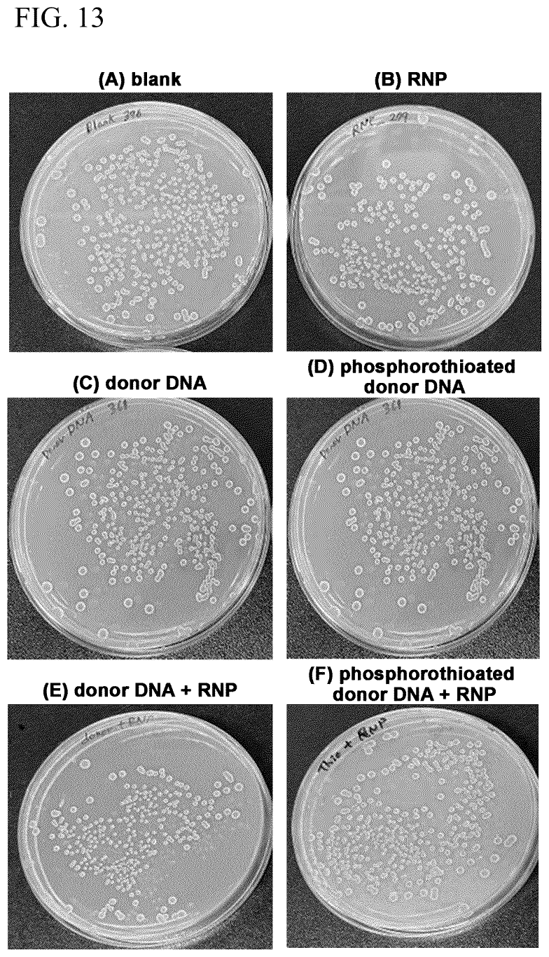

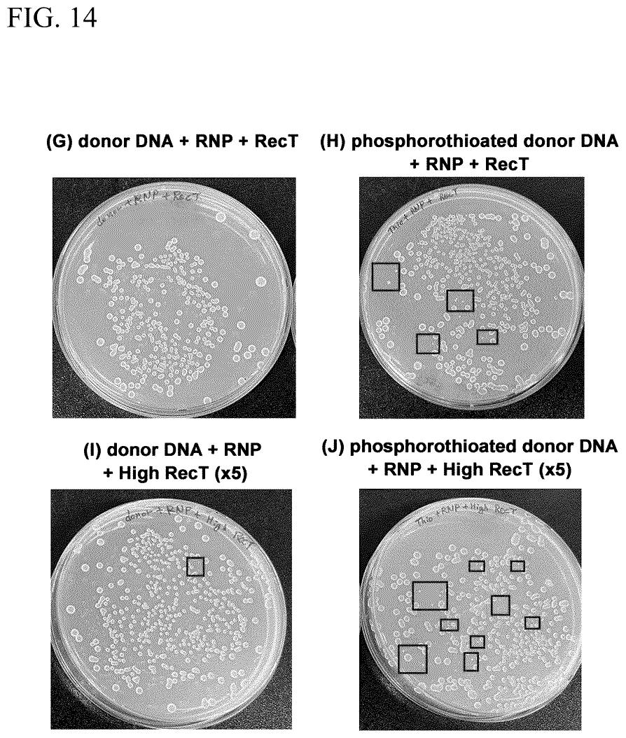

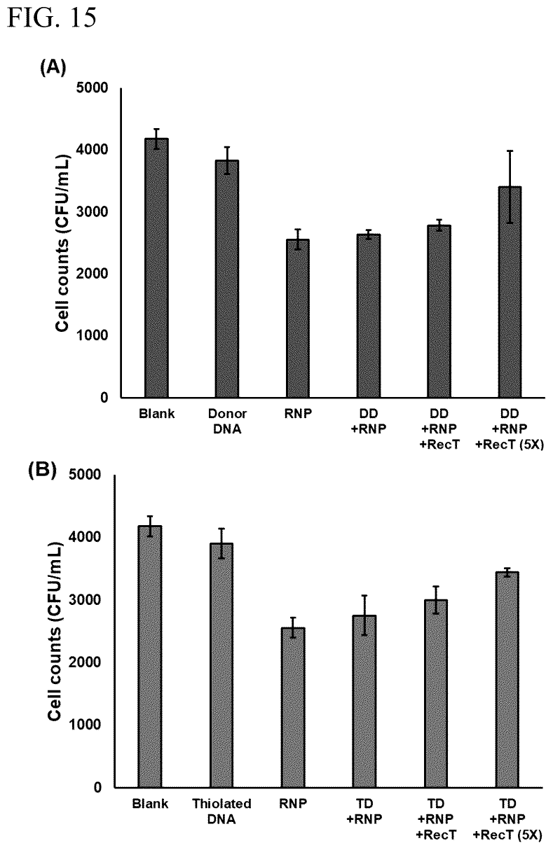

The transformed strains were inoculated onto phenylethyl alcohol agarose plates containing 2% sucrose and cultured (FIGS. 13 and 14). The concentration of viable cells that were completely transformed was then derived from the resulting culture (FIG. 15).

As can be seen from FIG. 15, when electroporation was performed after mixed with RNP, the number of cells was significantly reduced, but when electroporation was performed after further mixed with RecT, the number of dead cells was reduced, and the number of dead cells in the high concentration RecT treatment group was further reduced. The results show that RecT serves as a protector during the electric shock that occurred during the transformation process.

On the other hand, in mutant strains completely transformed, a non-functional DSR mutant gene was inserted in the middle of the DSR gene, causing the DSR gene to be knocked out. When the DSR gene is knocked out, dextransucrase is not expressed and thus dextran is not synthesized from sucrose. Dextran has a high molecular weight and is sticky, and forms a polysaccharide when secreted. In this example, the transformed strains could be screened using this property.

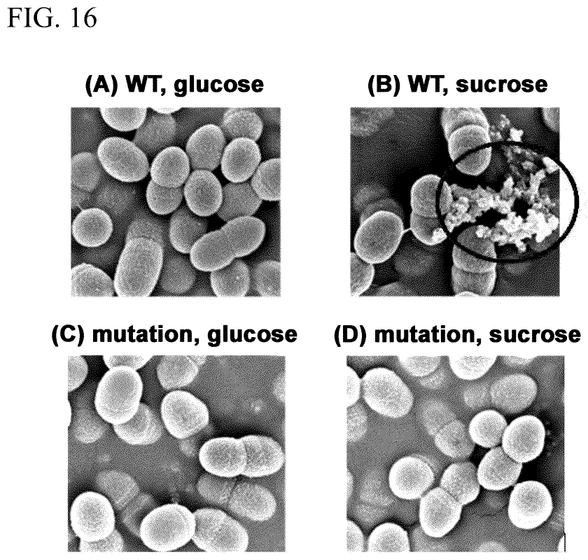

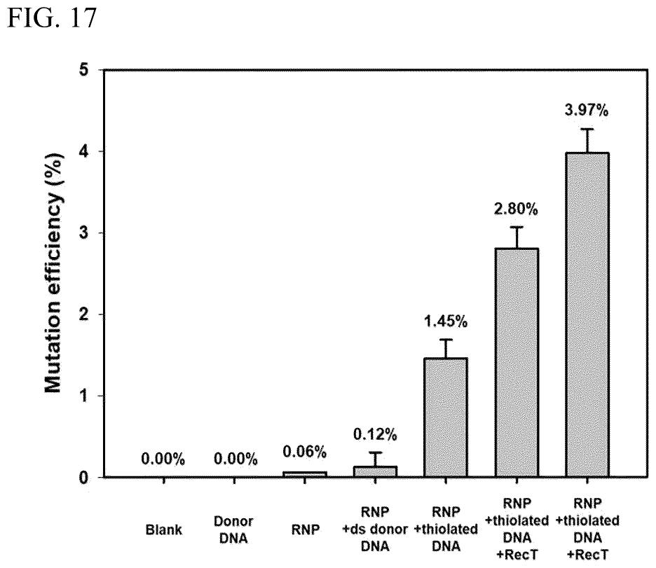

Specifically, the strains isolated by culturing on an agarose plate through Example 6-2 were observed by SEM to determine whether or not polysaccharides were formed (see FIG. 16), mutant strains were screened, and the transformation efficiency (number of mutant strains/number of viable cells) was calculated (Table 4, FIG. 17).

TABLE 4

| | Number of | Number of | |
|---|---|---|---|
| | viable | mutant | Mutation |
| | cells | strains | efficiency |
| Blank | 388 | 0 | 0 |
| Donor DNA | 340 | 0 | 0 |
| Phosphorothioated donor | 347 | 0 | 0 |
| DNA | | | |
| RNP | 234 | 0 | 0 |
| RNP + donor DNA | 267 | 2 | 0.74% |
| RNP + phosphorothioated | 214 | 2 | 0.93% |
| donor DNA | | | |
| RNP + phosphorothioated | 307 | 9 | 2.9% |
| donor DNA + RecT | | | |
| RNP + phosphorothioated | 355 | 13 | 3.7% |
| donor DNA + x5 RecT | | | |

This shows that the use of phosphorothioated donor DNA and RecT could increase the transformation efficiency.

### 6-4 Characterization of Transformed Strain

First, whether or not there was a problem with the screening method depending on whether or not polysaccharide was formed in Example 6-3 was determined.

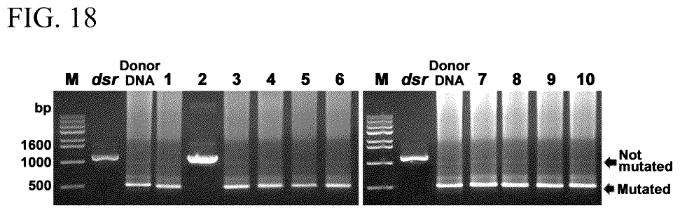

The dsr gene sequences of the wild-type strain and the mutant strain were PCR amplified using the custom oligomer 508 DSR-check-F (SEQ ID NO: 15, CACACCGATTTTTGTTTCAATTGCT) and 508 DSR-check-R (SEQ ID NO: 16, AAGCTCTACAAGTCTGGTAAGAGTTG) primers (FIG. 18).

As can be seen from FIG. 18, the wild-type strain had a band of 1,230 bp, while the mutant strain had a band amplified to 508 bp because the phosphorothioated donor DNA was fully inserted. Phosphorothioated DNA was inserted into 9 colonies, among the 10 colonies where polysaccharides were not formed, which indicates that screening mutant strains by determining whether or not polysaccharides were formed was completely performed with a very high probability.

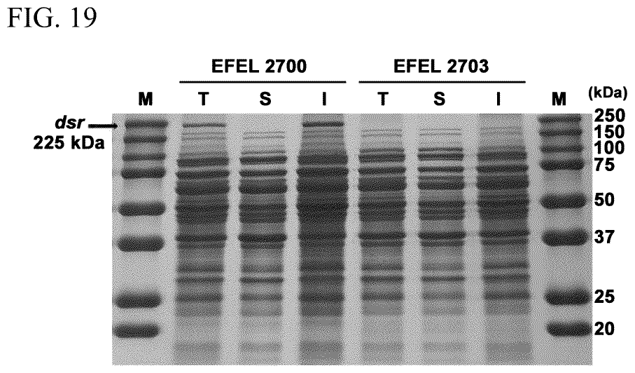

In order to compare the enzyme expression patterns, wild-type and mutant strains were cultured in MRS containing 2% sucrose. Then, the total cell protein profile was analyzed by SDS-PAGE (FIG. 19).

As can be seen from FIG. 19, 225 kDa dextransucrase is expressed in the wild-type strain (EFEL2700), but it is not expressed in the mutant strain (EFEL2703). The results indicate that the dextransucrase (DSR, EC 2.4.1.5)-expressing gene was successfully removed from the cells in the mutant strain.

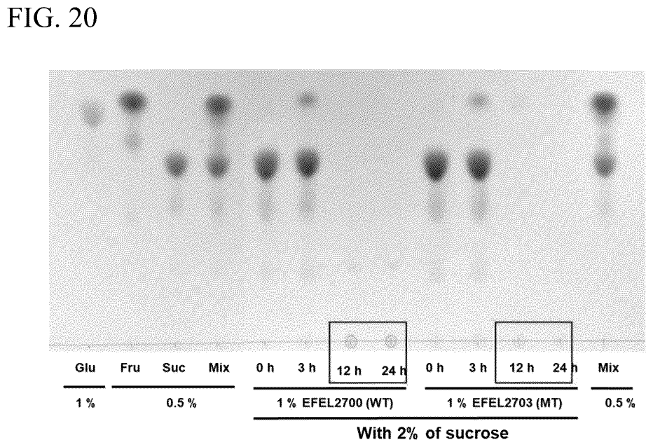

In order to compare the dextran formation between the wild type and mutant strains, the two strains were cultured in MRS medium containing 2% sucrose at 30° C. for 24 hours, and then TLC analysis was performed (FIG. 20).

As can be seen from FIG. 20, dextran polysaccharide was detected in the wild type strain even after 12 hours when sucrose was rapidly consumed, but dextran polysaccharide was not detected in the mutant strain after 12 hours. These results indicate that the dextransucrase gene (DSR, EC 2. 4.1.5) was successfully knocked out in the mutant strain.

### Example 7: Transformation of Bifidobacterium bifidum

In this example, transformation of Bifidobacterium bifidum BGN4 (KCCM12754P) was performed using the methods of Examples 1 to 6 and the efficiency of transformation was evaluated.

Meanwhile, after transformation, the original upp (uracil phosphoribosyltransferase) gene sequence (SEQ ID NO: 17) was amplified to 1,200 bp, while in the mutant strain, the gene sequence was amplified to 642 bp, which means that transformation was performed so that the upp gene was knocked out.

### 7-1. Production of Cas9/sgRNA RNP

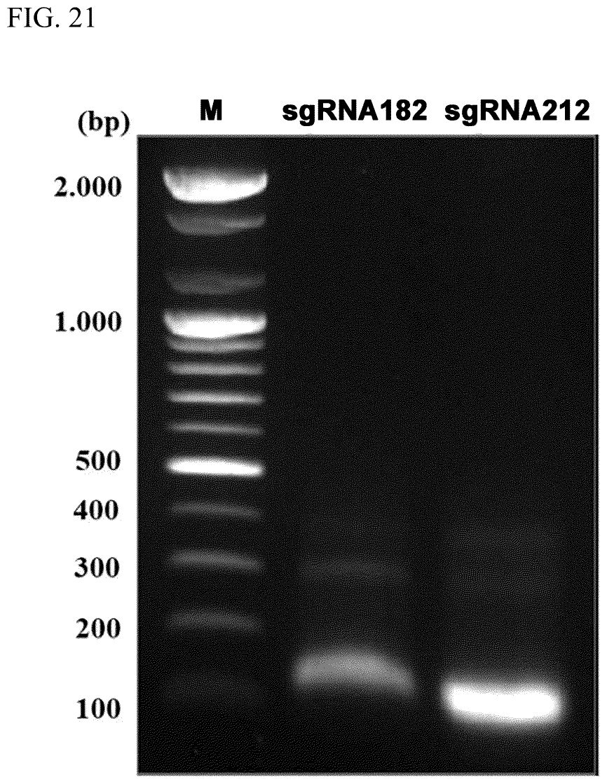

sgRNA was synthesized in the same manner as in Example 1-2, except that, the goal of editing the uracil phosphoribosyltransferase (upp) gene of Bifidobacterium bifidum BGN4, sgRNA was synthesized using the oligo primers in Table 5 below (FIG. 21).

TABLE 5

| sgRNA | | SEQ ID |
|---|---|---|
| primer | Sequence | NO. |
| Oligo | GAAATTAATACGACTC | 18 |
| F(upp182) | ACTATAGCGAAACCCC | |
| | CGTCGCCCCCAGTTTT | |
| | AGAGCTAGAAATAGCA | |
| | AG | |
| Oligo | GAAATTAATACGACTC | 19 |
| F(upp212) | ACTATAGGCGAAGGAC | |
| | GGGAACGATGAGTTTT | |
| | AGAGCTAGAAATAGCA | |
| | AG | |
| Oligo | AAAAAAGCACCGACTC | 20 |
| R(common) | GGTGCCACTTTTTCAA | |
| | GTTGATAACGGACTAG | |
| | CCTTATTTTAACTTGC | |
| | TATTTCTAGCTCTAAA | |
| | AC | |

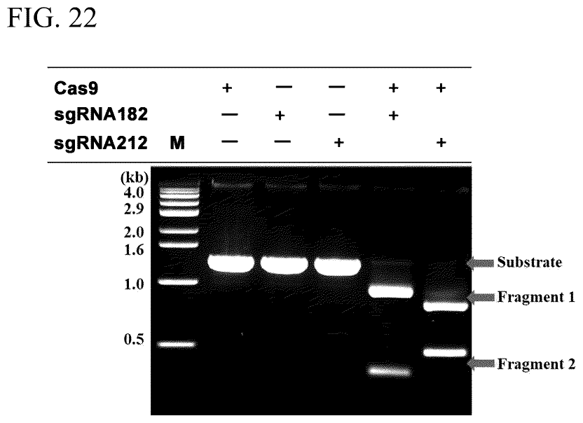

Synthetic sgRNA and cas9 produced in Example 1-1 were mixed at a molar ratio of 1:1 to assemble Cas9/sgRNA RNP. Then, when the Cas9/sgRNA RNP was reacted with PCR amplicon produced using gDNA of Bifidobacterium bifidum BGN4 as a template, RNP produced using sgRNA182 and sgRNA212 exhibited complete activity (FIG. 22).

### 7-2. Production of Phosphorothioated Donor DNA

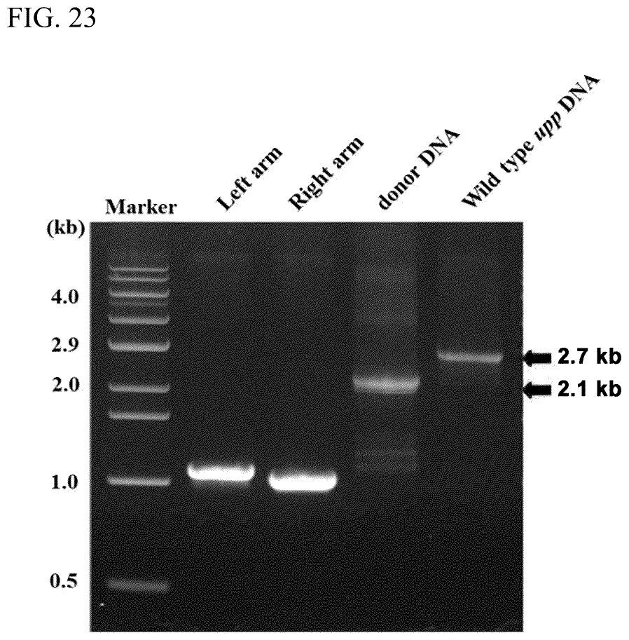

Donor DNA and phosphorothioated donor DNA were produced in the same manner as in Example 3, except that upstream primers (Upp-U-F/Upp-U-R/phosphorothioated Upp-U-F) and downstream (Upp-D-F/Upp-D-R/phosphorothioated Upp-D-R) primers shown in Table 6 were produced such that a part of the Upp gene was deleted, and then were linked to each other by overlap extension PCR to produce donor DNA and phosphorothioated donor DNA (FIG. 23).

As can be seen from FIG. 23, when the existing upp gene was amplified, a band appeared at 2.7 kb, whereas, in donor DNA and phosphorothioated donor DNA, a band appeared at 2.1 kb, indicating successful production.

TABLE 6

| | | SEQ ID |
|---|---|---|
| Name | Sequence | NO. |
| Upp-U-F | TGCCCGCGTTGTAT | 21 |
| | TTCGAGGT | |
| Upp-U-R | GTCCACCGACTGTT | 22 |
| | CCCAGACTGTAGCT | |
| | CTCGACCCCTCCAT | |
| | CGCCTTCAA | |
| Upp-D-F | AGAGCTACAGTCTG | 23 |
| | GGAACAGTCGGTGG | |
| | ACGTTCGGCACGAT | |
| | GACCGTCGA | |
| Upp-D-R | GCCACCATGCAACC | 24 |
| | AGCGAATC | |
| Phosphorothioated | *T*G*CCCGCGTT | 25 |
| Upp-U-F | GTATTTCGAGGT | |
| Phosphorothioated | *G*C*CACCATGC | 26 |
| Upp-D-R | AACCAGCGAATC | |

*means phosphorothioated modification between nucleotides

### 7-3. Cell Preparation and Transformation

B. bifidum BGN4 cells were cultured in MRS medium containing 0.05% L-cysteine HCl (Sigma-Aldrich) to induce activation of the B. bifidum BGN4 cells. The activated strains were cultured at 37° C. under anaerobic conditions until the OD₆₀₀ reached 0.4 to 0.5. At this time, 0.2 M NaCl was added to the medium to weaken the cell wall and cultured. Then, the cells were harvested by centrifugation and a resuspended at concentration of 10^{4˜5}/mL in an electroporation solution (consisting of 0.5 M sucrose and 1 mM ammonium citrate, pH 6.0) to prepare competent cells. Meanwhile, the competent cells were stored at −80° C. and used. Then, 20 μL of the B. bifidum BGN4 competent cell solution prepared above was mixed with RNP (Cas9:sgRNA=100 μg:30 μg=1:1 molar ratio), RecE 26 μg, RecT 24 μg, and phosphorothioated donor DNA 25 μg, and transformation was performed by electroporation ((Electroporation; 10 kV cm⁻¹, 25 μF, 400Ω).

### 7-4. Confirmation of Transformation Efficiency

The UPP (uracil phosphoribosyltransferase) gene in the mutated strain was knocked out. Although 5-FU (5-fluorouracil) was contained in the medium, it was not activated and thus no toxicity was observed. In this example, the mutant strain was screened using this property.

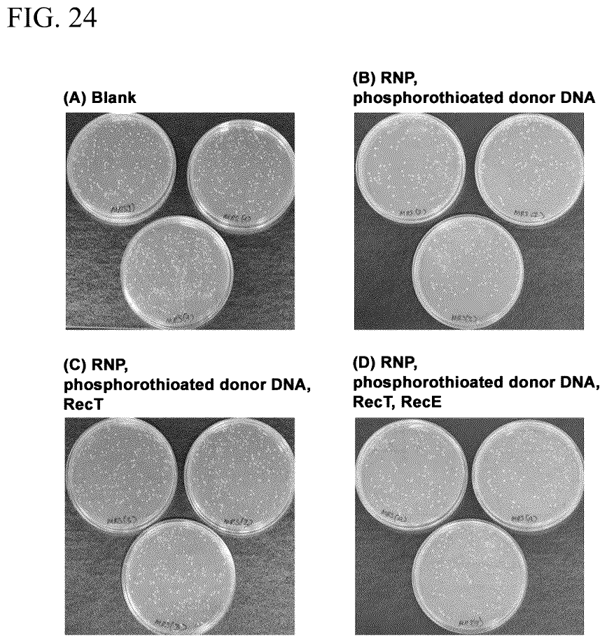

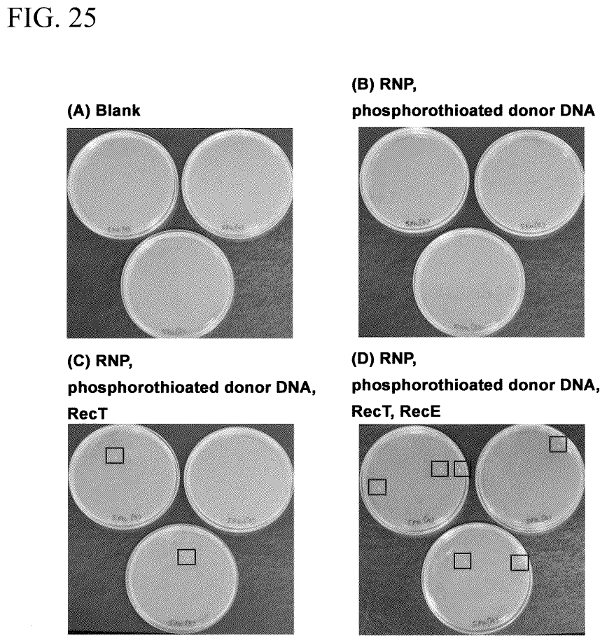

Specifically, the transformed cells were cultured under anaerobic conditions for 72 hours. The cells were plated and cultured on MRS medium containing L-cysteine or MRS medium containing 5-FU (5-fluorouracil) containing L-cysteine (FIGS. 24 and 25).

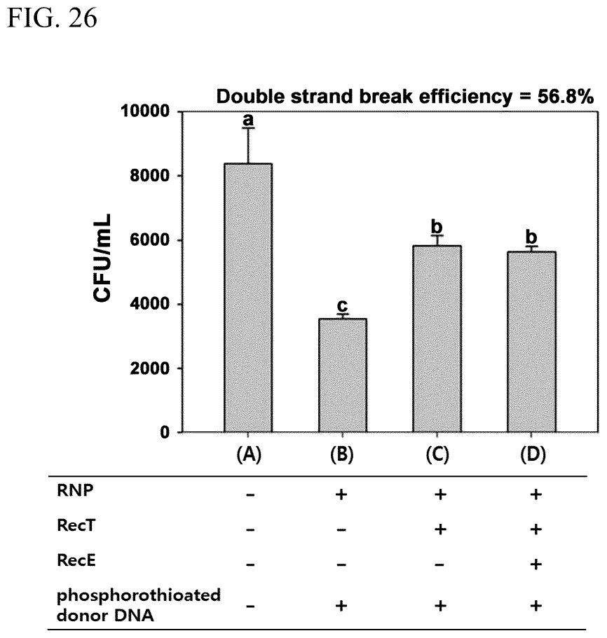

Then, the concentration of viable cells was determined using FIG. 24, and the difference in survival rate between the control group (A) and the group into which RNP and donor DNA were introduced (B) was calculated (FIG. 26).

When DNA was cleaved due to the endonuclease activity of RNP, the cells died. This indicates that DNA cleavage efficiency was 56.8%.

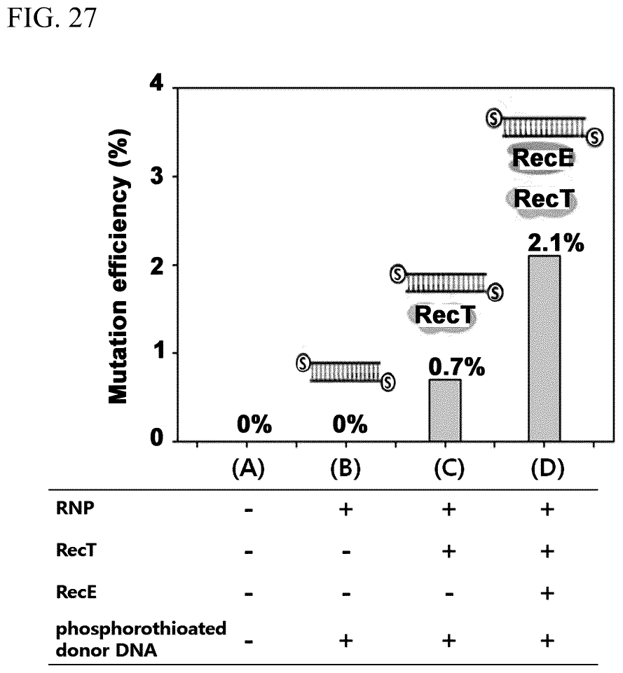

The number of viable cells surviving in the MRS medium containing cysteine and 5-FU (5-fluorouracil) was measured using FIG. 25, and the transformation efficiency was calculated using the number of viable cells (FIG. 27).

When neither RecT nor RecE was introduced, no mutations occurred. When RecT was further introduced, a mutation efficiency of 0.7% was observed. When both RecE/T were introduced, the transformation efficiency (value obtained by dividing the viable cell concentration on the MRS medium plate containing 5-FU by the viable cell concentration on the MRS agar plate) increased to 2.1%. This means that the RecE/T recombinant protein plays an essential role in transforming Gram-positive bacteria or lactic acid bacteria.

### 7-5. Characterization of Transformed Strain

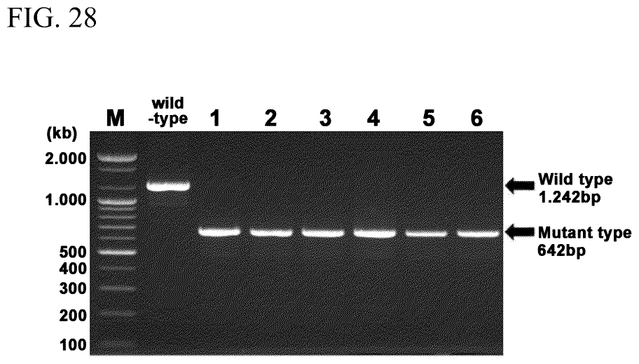

Whether or not the transformation was complete was determined. The Bifidobacterium bifidum BGN4 mutant strain screened from the process of Example 7-4 was grown in MRS broth containing 0.05% L-cysteine·HCl and 5-FU at 37° C. under anaerobic conditions. Then, the cells were centrifuged at 10,000×g for 1 minute and chromosomal DNA was extracted using the genomic DNA (gDNA) prep kit from Solgent. Then, whether or not the mutation was complete was determined using the Upp-check-F primer (SEQ ID NO: 27, ATGATGTCGAGCTTGCAGTAGC) and the Upp-check-R primer (SEQ ID NO: 28, CTTTCGCCTCGGACCCGTAC) for amplification (FIG. 28). This shows that the original sequence was amplified to 1242 bp, while the mutant strain was amplified to 642 bp, indicating that the transformation occurred completely.

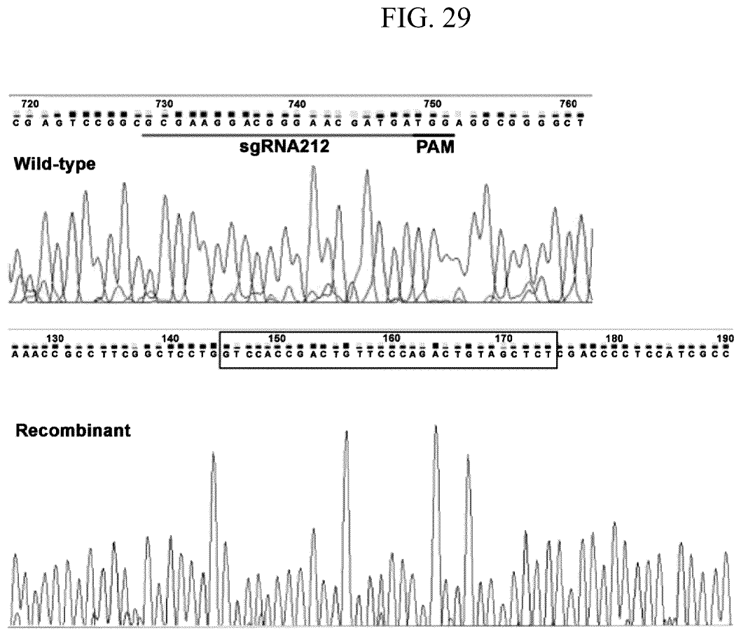

In addition, amplicon sequencing was performed to verify this more clearly. The genomic DNA of the mutant strain was analyzed by Cosmogentech. Then, the obtained sequence was compared with the wild-type reference sequence to determine whether or not there were any sequence changes or mutations (FIG. 29).

The result of the sequence analysis showed that the PAM sequence (5′-TGG-3′) was deleted from the upp gene of the wild-type strain in the obtained mutant strain, and the additionally introduced phosphothioated 30 bp homologous sequence was inserted, so that the upp gene was successfully knocked out in the mutant strain.

As is apparent from the above description, the present invention provides a method of synthesizing sgRNA and Cas9 protein separately outside the cells (in vitro), binding the two compounds to form a ribonucleoprotein complex (RNP) in a test tube, and then injecting the ribonucleoprotein complex (RNP) into target cells. However, there is a problem in which it is difficult to recombine genes of Gram-positive bacteria and lactic acid bacteria even using the CRISPR/Cas system due to the cell wall structure thereof. On the other hand, the present invention demonstrated that genes of lactic acid bacteria and gram-positive bacteria that are difficult to recombine can be recombined with high efficiency using recombinases in combination with a phosphorothioated donor DNA in an RNP recombination system using Cas protein.

In the present invention, Leuconostoc citreum, which is a major lactic acid bacterium in kimchi, and Bifidobacterium bifidum, which is a major lactic acid bacterium in yogurt, were used as verification cases of the present invention. Both of these are lactic acid bacteria and belong to the gram-positive bacteria with thick cell walls. In Leuconostoc citreum, according to the technology of the present invention, the dextran sucrase gene (dsr) that produces dextran polysaccharide was knockout to select a mutant strain that does not produce slime on a plate medium containing sucrose. In addition, in Bifidobacterium bifidum, the uracil phosphoribosyl transferase (upp) gene involved in DNA synthesis was knockout to select a mutant strain that grows on a plate medium containing 5-fluorouracil (5-FU) (counter selection).

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### SEQUENCE LISTING FREE TEXT

An electronic file is attached.

What is claimed is:

1. A method of recombining genes in Gram-positive bacteria cells by introducing a ribonucleoprotein (RNP) complex in which a site-specific endonuclease and a guide RNA (gRNA) that specifically binds to a target DNA and guides a cleavage site of the site-specific endonuclease are linked, and a donor DNA into the Gram-positive bacteria cells,
wherein recombinases are introduced into the cells along with the ribonucleoprotein and the DNA donor, and the DNA donor has a phosphorothioate structure in which at least one oxygen of the phosphate backbone structure is substituted with sulfur.

2. The method according to claim 1, wherein the site-specific endonuclease comprises one selected from Cas3, Cas9, Cas12, Cas12a (Cpf1), Cas13, Cas14, and nickase variants thereof.

3. The method according to claim 1, wherein the recombinant enzyme comprises one or more selected from RecT, RecE, RedE, RedT, RamdaE, and RamdaT.

4. The method according to claim 1, wherein the ribonucleoprotein (RNP) comprises the site-specific endonuclease and the guide RNA (gRNA) in a molar ratio of 1:0.8 to 1:1.2.

5. The method according to claim 1, wherein the cell is a competent cell that has a weakened cell wall due to being cultured along with at least one selected from penicillin, ethanol, glycine, and sodium chloride (NaCl).

6. The method according to claim 1, wherein the cell is a lactic acid bacterium.

7. The method according to claim 1, wherein the DNA donor has a phosphorothioate structure in which oxygen of the phosphate backbone structure at both ends is substituted with sulfur.

8. The method according to claim 1, wherein the introduction is performed by electroporation.

9. The method according to claim 8, wherein the electroporation is performed at a voltage of 8 to 12 kV/cm.

10. A cell genetically recombined by the method according to claim 1.


SEQUENCE LISTING (28 sequences)

SEQ ID NO: 1 - DNA, 4518 bp, Leuconostoc citreum
  source 1..4518: mol_type = genomic DNA
ttgattaaagagagaaatgtacgaaaaaagctctacaagtctggtaagagttgggttattgggggactcattttatcgacaattatgctgtctatgaccgctacttcacaaaatgttaatgcagatagcacaaacacagtgacggataagtcagttactgtctccaataattcgaatacaaccaatcaacacgatactgtcgttgacaaacaaacgacacctgtcaaaaatgaccaaacaacacaacaaatcgccgcaaatgccacccaagcagaaaaagtaaaagcatcagacacaacgactgatacgcaaaagcaagctgaaacggcaaacaacactaacaaggattcgatagataatctcaccaagcagttgccgactgttacgccaacagctaatcaaaaaactggttatctggaaaaagatggtaaatggtactatgtaaccagtgataacacacttgctaaggggttgactactgttgacaaccacaagcagtattttgacaacaatggcgtgcaggcaaaagtccaattcgttactgataacagtaaaacatactatctcgattctaactccggtaacgcagtaaccgggatacaacaaattggctcacaaacattagccttcaatgacaacggtgaacaagtttttgctgatttctatacagcgccagatggcaaaacttattattttgacgataaaggacaagcaactattggtctaaaggccattaatgggcacaattattacttcgatagtttgggacaactaaaaaaaggatttaccggtgtcattgacggtcaagtacgctattttgatcaagaatcaggacaagaggtatcaacaaccgactcacaaatcaaagaaggtttaacttctcagacaacagactatacagcacataatgccgttcacagcaccgatagcgctgatttcgacaattttaatggttatttgaccgcttcttcatggtatcgccctaaagatgttttaagaaatggtcaacactgggaagcaacaacagctaatgacttccggcccattgtgtcagtttggtggcctagcaagcaaacacaagtaaattacctaaactacatgtctcaaatgggactcattgacaatcgtcagatgttctcgctaaaagacaatcaagccatgttgaatattgcttgcacaacagtccaacaagcaattgaaacaaaaatcggtgtggctaatagtacagcatggcttaaaacagccattgatgatttcattcgtacacagccacaatggaacatgtcgagtgaagatcccaaaaatgatcatttacaaaacggcgctttgactttcgtcaacagtccattgacaccagatactaactctaatttcagactattaaatcgcacaccaacaaaccagacaggtgtgccaaaatatacaattgatcaatctaagggtggttttgaactcttactcgctaatgatgtagacaactctaatcctgttgtgcaagctgagcagttaaattggttacactatttgatgaattttggtagcattacagcaaacgattctgctgctaattttgatgggatacgtgtcgatgctgtcgataatgttgacgctgatttgctccagattgcagcagattatttcaaagctgcttatggtgttgataaaaatgacgcaacagcaaatcaacatctttcaattcttgaagattggagccataacgaccctgaatacgtgaaggattttggtaataatcaactcacaatggatgattacatgcatacccagttaatctggtcgttgactaaagatatgcgtatgcgtggtaccatgcaacgcttcatggactattacctcgtcaatcgcaatcacgatagtaccgaaaacactgccattccaaattacagctttgttcgcgcacacgatagtgaagtacaaacagtcattgctcaaattatttctgagttacatcccgacgtaaaaaatagtttggcaccaacagcagaccagctagccgaagcctttaaagtttataataacgatgaaaaacaggcggataagaaatatacacaatacaacatgcctagcgcctatgcgatgctgttaactaataaagatacagtaccgcgcgtttattatggtgatttatacaccgatgatggtcaatatatggcaaataagtccccttattttgatgccatcaacggcttgctaaagtcacgtatcaaatatgttgctggtggtcagtcaatggctgttgatcaaaacgatatcctgacaaatgttcgttatggtaaaggtgccatgagtgtgacagatagcggtaatgcagacacacgaacacaaggtattggtgtgattgtcagtaataaagaaaatctggccttaaaatcaggcgacacggtgacattacacatgggtgccgctcacaaaaatcaagcattcagattattattagggacaactgctgacaatttgtcttattatgataatgacaacgccccagtaaagtacaccaatgatcagggcgatttaatttttgataatactgaaatctatggtgtccgtaacccgcaagtctctggcttcttagctgtttgggtgcctgttggggctgacagccatcaagacgcgcgtactttgtctgacgacacagcccatcatgatggcaaaaccttccactcaaatgctgctttagattctcaggttatttacgaaggtttttcaaatttccaagcttttgccacaaacactgaagactatacaaatgctgtcattgcaaaaaatggtcagttattcaaagattggggtatcacaagtttccagttggcaccacaatatcgttcaagcaccgataccagtttcttagattcaattatccaaaatggttatgcctttacagatcgttatgatttgggctacggtacaccaacaaaatatggcacagttgaccagttacgcgatgccatcaaggctctgcacgcaaatggcatccaagcaatcgctgactgggtacccgaccaaatttataatttaccgggtcaagaattagcgactgtcacacgaacaaactcttatggtgataaagacactaactcagatattgatcagtcactatatgtcatacaaagtcgtggtggtggtaaataccaagcacagtatggcggtgccttcttatctgatatccagaaaaaatatccagcacttttcgaaacaaaacaaatttctacagggctacctatggatcctagtcagaaaataacagaatggtctggtaaatactttaatggctcaaatattcaaggcaaaggggctggctatgtcttaaaagacagtggtaccgatcaatactataaggttacatcaaacaataataatcgtgacttcttaccaaaacagttaacagatgacttatctgaaacaggatttgtccgcgataacattggtatggtctactacacattgagtggctatctagctcgaaacacctttatacaagatgataatggcaattattattactttgatagcaccggccatctcgttactggcttccagaatattaataaccatcactatttcttcctaccaaacggtattgaactcgttcaatctttcttacagaatgctgacggttcaacgatttattttgaccaaaaagggcgtcaagtatttaatcaatacattactgaccaaaccggtaccgcctattacttccagaatgatggcacaatggtcacttctggcttcactgaaatcgatggtcataagcaatacttctacaagaacggcacacaagtcaaagggcaatttgtatcagacactgatggtcacgttttctacttagaagctggtaacggcaacgtggcgacacaaagatttgcacaaaatagtcaaggtcagtggttctatttgggtaatgatggcattgccttgactggtttgcaaacaatcaatggtgttcaaaattatttctacgccgatggtcatcaaagtaagggtgattttattacgatacaaaatcacgtattatatactaacccactaactggcgctataacgacaggtatgcaacaaattggtgacaagatttttgtctttgacaatacgggcaacatgttgaccaatcaatactatcaaacactagatggccaatggttacatttaagcactcaaggtccagcagacactggtttggtaaacattaatggtaatttgaaatatttccaagctaatggtcggcaagtgaaaggtcaatttgtgactgatcctatcacgaacgtgagttattatatgaatgccactgatggttcggcagtatttaatgactactttacctatcaaggccaatggtatttaacggatagtaattatcaactcgtcaaaggatttaaagttgttaataataagctacaacattttgatgaaataacaggcgtacaaactaaatcagctcatatcatcgttaataatcgaacatacattttcgatgaccaaggttactttgtctcagtcgcttaa

SEQ ID NO: 2 - DNA, 66 bp, synthetic construct
  source 1..66: mol_type = other DNA
gaaattaatacgactcactatagtctacaagtctggtaagagtgttttagagctagaaatagcaag

SEQ ID NO: 3 - DNA, 66 bp, synthetic construct
  source 1..66: mol_type = other DNA
gaaattaatacgactcactatagaacgacagtatcgtgttgatgttttagagctagaaatagcaag

SEQ ID NO: 4 - DNA, 66 bp, synthetic construct
  source 1..66: mol_type = other DNA
gaaattaatacgactcactatagcgtaacagtcggcaactgctgttttagagctagaaatagcaag

SEQ ID NO: 5 - DNA, 66 bp, synthetic construct
  source 1..66: mol_type = other DNA
gaaattaatacgactcactatagatctggaaaaagatggtaaagttttagagctagaaatagcaag

SEQ ID NO: 6 - DNA, 82 bp, synthetic construct
  source 1..82: mol_type = other DNA
aaaaaagcaccgactcggtgccactttttcaagttgataacggactagccttattttaacttgctatttctagctctaaaac

SEQ ID NO: 7 - DNA, 888 bp, Lactobacillus plantarum
  source 1..888: mol_type = genomic DNA
atgagtaatgagctagttacgatggttaataacaatattgaggatatgaagaataatgaaggcttgtcattaccacctgattattcagtagggaatgcattaaacagtgcttacttgattttgagtgatacgtctaagggccaaccattacttgataagtgtgaccaaggatcagttatcaaggcgttaatgaacatggcaattcaaggattgagcccagctaaaaaccaatgctatttcattccttatggtaaccagttagtcatgcagcgttcctattttggctcaattagcgttgtaaagcgtctttcaaacgttaaggatattcaggcacaggttgtccacaaagacgacacgttcaagattggtggtaaagatggagtgttggtggttaaagagttcgagccaagctttgagaacctagataagccaattatcggggcctttgcatggatcgaagacatcaacgggaaccggacatacacggttatgacaaaaaaggacatcgacatcagttggagccacgctaagacgaagaaggttcaaaacgagttcccagaagagatggctaaacggactgtaattaatcgagctgctaagttctatattaattcaagctcagacaacgatttatttgtaaaagcagttaacgaaaccactagcaacgaatatgagaatgacgaaagaaaagacgtaacaccggctaagcggtcactagtagctgacgtagcagagaataaagccgagaaggtagaatctgccgaaccagctaaagaacccgttagaacggctgtaaaggaggcatcaagcaatgatcaagaacctgtcaaaggcgaagtcgaccagcaaaacctcttcgacaacctcggagactttgacgccagctaa

SEQ ID NO: 8 - DNA, 975 bp, Escherichia coli
  source 1..975: mol_type = genomic DNA
atgggcagcagccatcatcatcatcatcacagcagcggcctggtgccgcgcggcagccatatggaacatccgcacaatgagaatgctggcagcgatccgcatcgcgattgcagtgatgaaactggcgaagtcgcagatcccgtaatcgtagaagacatagagccaggtatttattacggaatttcgaatgagaattaccacgcgggtcccggtatcagtaagtctcagctcgatgacattgctgatactccggcactatatttgtggcgtaaaaatgcccccgtggacaccacaaagacaaaaacgctcgatttaggaactgctttccactgccgggtacttgaaccggaagaattcagtaaccgctttatcgtagcacctgaatttaaccgccgtacaaacgccggaaaagaagaagagaaagcgtttctgatggaatgcgcaagcacaggaaaaacggttatcactgcggaagaaggccggaaaattgaactcatgtatcaaagcgttatggctttgccgctggggcaatggcttgttgaaagcgccggacacgctgaatcatcaatttactgggaagatcctgaaacaggaattttgtgtcggtgccgtccggacaaaattatccctgaatttcactggatcatggacgtgaaaactacggcggatattcaacgattcaaaaccgcttattacgactaccgctatcacgttcaggatgcattctacagtgacggttatgaagcacagtttggagtgcagccaactttcgtttttctggttgccagcacaactattgaatgcggacgttatccggttgaaattttcatgatgggcgaagaagcaaaactggcaggtcaacaggaatatcaccgcaatctgcgaaccctgtctgactgcctgaataccgatgaatggccagctattaagacattatcactgccccgctgggctaaggaatatgcaaatgactaa

SEQ ID NO: 9 - DNA, 26 bp, synthetic construct
  source 1..26: mol_type = other DNA
cagctaaactcactttaactattgct

SEQ ID NO: 10 - DNA, 54 bp, synthetic construct
  source 1..54: mol_type = other DNA
gtccaccgactgttcccagactgtagctctgtcgttgtgtctgatgcttttact

SEQ ID NO: 11 - DNA, 54 bp, synthetic construct
  source 1..54: mol_type = other DNA
agagctacagtctgggaacagtcggtggacgaagcaacaacagctaatgacttc

SEQ ID NO: 12 - DNA, 25 bp, synthetic construct
  source 1..25: mol_type = other DNA
ctaggcatgttgtattgtgtatatt

SEQ ID NO: 13 - DNA, 26 bp, synthetic construct
  source 1..26: mol_type = other DNA
cagctaaactcactttaactattgct

SEQ ID NO: 14 - DNA, 25 bp, synthetic construct
  source 1..25: mol_type = other DNA
ctaggcatgttgtattgtgtatatt

SEQ ID NO: 15 - DNA, 25 bp, synthetic construct
  source 1..25: mol_type = other DNA
cacaccgatttttgtttcaattgct

SEQ ID NO: 16 - DNA, 26 bp, synthetic construct
  source 1..26: mol_type = other DNA
aagctctacaagtctggtaagagttg

SEQ ID NO: 17 - DNA, 642 bp, Bifidobacterium bifidum
  source 1..642: mol_type = genomic DNA
atggaacttcacgtactcgaccacccgctggtggaacacaagctcaccgtcctgcgcgacaaaaacacctcatccgccactttccgcgagctcgtcaccgaactcgtcatgctcgaagcctacgaggcgacccgcaacctcgacatcgtcgacaagcccatcgaaacccccgtcgcccccatggtcggcaagcacatcgccagccccgcccccatcatcgttcccgtccttcgcgccggactcggcatgctcgacggcatgaccaaaatgatccccaccgccgaagtcggcttcctcggcatgaagcgcgacgaagagcaccccacccagcagatcacttacgccaaccgtctccccgaagacctcaccggccgtcagtgcttcctcatcgaccccatgctcgccaccggcggcaccctcgtcaccgccacccactacctcgccgagcgcggcgccaaagacgtcaccgccatcaacatcatcgccgcccccgaaggcatccaatacgtccaagaccacatcgacccctccatcgccttcaaggtcgtcgtctgcgccgtcgatgaaaaactcaacgaaaaggcgtacattgtgccggggcttggcgacgccggcgaccgcctctacggcgtcattgattga

SEQ ID NO: 18 - DNA, 66 bp, synthetic construct
  source 1..66: mol_type = other DNA
gaaattaatacgactcactatagcgaaacccccgtcgcccccagttttagagctagaaatagcaag

SEQ ID NO: 19 - DNA, 66 bp, synthetic construct
  source 1..66: mol_type = other DNA
gaaattaatacgactcactataggcgaaggacgggaacgatgagttttagagctagaaatagcaag

SEQ ID NO: 20 - DNA, 82 bp, synthetic construct; identical to SEQ ID NO: 6
  source 1..82: mol_type = other DNA

SEQ ID NO: 21 - DNA, 22 bp, synthetic construct
  source 1..22: mol_type = other DNA
tgcccgcgttgtatttcgaggt

SEQ ID NO: 22 - DNA, 51 bp, synthetic construct
  source 1..51: mol_type = other DNA
gtccaccgactgttcccagactgtagctctcgacccctccatcgccttcaa

SEQ ID NO: 23 - DNA, 51 bp, synthetic construct
  source 1..51: mol_type = other DNA
agagctacagtctgggaacagtcggtggacgttcggcacgatgaccgtcga

SEQ ID NO: 24 - DNA, 22 bp, synthetic construct
  source 1..22: mol_type = other DNA
gccaccatgcaaccagcgaatc

SEQ ID NO: 25 - DNA, 22 bp, synthetic construct
  source 1..22: mol_type = other DNA
tgcccgcgttgtatttcgaggt

SEQ ID NO: 26 - DNA, 22 bp, synthetic construct
  source 1..22: mol_type = other DNA
gccaccatgcaaccagcgaatc

SEQ ID NO: 27 - DNA, 22 bp, synthetic construct
  source 1..22: mol_type = other DNA
atgatgtcgagcttgcagtagc

SEQ ID NO: 28 - DNA, 20 bp, synthetic construct
  source 1..20: mol_type = other DNA
ctttcgcctcggacccgtac